(19) United States
(12) Patent Application Publication
(10) Pub. No.: US 2024/0021310 A1
(43) Pub. Date: Jan. 18, 2024

(54) Data Transformations to Create Canonical Training Data Sets

(71) Applicant: Google LLC, Mountain View, CA (US)

(72) Inventors: Farhana Bandukwala, Mountain View, CA (US); Peter Brune, Mountain View, CA (US); Fanyu Kong, Mountain View, CA (US); David Roger Anderson, West Lakeville, MN (US)

(73) Assignee: Google LLC, Mountain View, CA (US)

(21) Appl. No.: 18/349,945

(22) Filed: Jul. 10, 2023

Related U.S. Application Data
(60) Provisional application No. 63/368,180, filed on Jul. 12, 2022.

(51) Int. Cl. G16H 50/20

(52) U.S. Cl. CPC G16H 50/20

(57) ABSTRACT

A method includes obtaining a dataset that includes health data in a Fast Healthcare Interoperability Resources (FHIR) standard. The health data includes a plurality of healthcare events. The method includes generating, using the dataset, an events table that includes the plurality of healthcare events and is indexed by time and a unique identifier per patient encounter. The method also includes generating, using the dataset, a traits table that includes static data and is indexed by the unique identifier per patient encounter. The method includes training a machine learning model using the events table and the traits table and predicting, using the trained machine learning model and one or more additional healthcare events associated with a patient, a health outcome for the patient.

### CROSS REFERENCE TO RELATED APPLICATIONS

This U.S. patent application is a continuation of, and claims priority under 35 U.S.C. § 119(e) to U.S. Provisional Application 63/368,180, filed on Jul. 12, 2022. The disclosure of this prior application is considered part of the disclosure of this application and is hereby incorporated by reference in its entirety.

### TECHNICAL FIELD

This disclosure relates to using data transformations to create canonical training data sets.

### BACKGROUND

Metrics for healthcare patients over time (e.g., regular readings of blood pressure, heart rate, sodium/glucose levels, etc.) are routinely used by clinicians to identify at-risk persons. As sensors get more numerous and more data is shared across institutions, clinicians have to sift through increasing amounts of data to understand the trends and identify the probability of “individualized” patient outcomes. Additionally or alternatively, hospital administrators are tracking operational and quality of care metrics such as length of stays, supply of equipment, staffing levels, etc. The end goal is to calculate the probability of a future positive or negative outcome such that timely interventions can be implemented.

### SUMMARY

One aspect of the disclosure provides a method for transforming data to create canonical training data sets for machine learning models. The method, when executed by data processing hardware, causes the data processing hardware to perform operations. The operations include obtaining a dataset that includes health data in a Fast Healthcare Interoperability Resources (FHIR) standard. The health data includes a plurality of healthcare events. The operations include generating, using the dataset, an events table that includes the plurality of healthcare events. The events table is indexed by time and a unique identifier per patient encounter. The method includes generating, using the dataset, a traits table that includes static data. The traits table is indexed by the unique identifier per patient encounter. The method also includes training a machine learning model using the events table and the traits table and predicting, using the trained machine learning model and one or more additional healthcare events associated with a patient, a health outcome for the patient.

Implementations of the disclosure may include one or more of the following optional features. In some implementations, obtaining the dataset includes receiving a training request defining a data source of the dataset and retrieving the dataset from the data source. Optionally, the operations further include normalizing one or more codes of the health data. In some examples, the operations further include normalizing one or more units of the health data.

The dataset may include a comma-separated values file. In some implementations, the traits table includes patient demographics. The events table may represent the dataset as a structured time-series. In some examples, the dataset includes nested data. In some examples, the operations further include generating a user-configurable trait table that includes context-specific static features indexed by the unique identifier per patient encounter. In some of these examples, generating the user-configurable trait table includes receiving the context-specific static features from a user.

Another aspect of the disclosure provides a system for transforming data to create canonical training data sets for machine learning models. The system includes data processing hardware and memory hardware in communication with the data processing hardware. The memory hardware stores instructions that when executed on the data processing hardware cause the data processing hardware to perform operations. The operations include obtaining a dataset that includes health data in a Fast Healthcare Interoperability Resources (FHIR) standard. The health data includes a plurality of healthcare events. The operations include generating, using the dataset, an events table that includes the plurality of healthcare events. The events table is indexed by time and a unique identifier per patient encounter. The method includes generating, using the dataset, a traits table that includes static data. The traits table is indexed by the unique identifier per patient encounter. The method also includes training a machine learning model using the events table and the traits table and predicting, using the trained machine learning model and one or more additional healthcare events associated with a patient, a health outcome for the patient.

This aspect may include one or more of the following optional features. In some implementations, obtaining the dataset includes receiving a training request defining a data source of the dataset and retrieving the dataset from the data source. Optionally, the operations further include normalizing one or more codes of the health data. In some examples, the operations further include normalizing one or more units of the health data.

The dataset may include a comma-separated values file. In some implementations, the traits table includes patient demographics. The events table may represent the dataset as a structured time-series. In some examples, the dataset includes nested data. In some examples, the operations further include generating a user-configurable trait table that includes context-specific static features indexed by the unique identifier per patient encounter. In some of these examples, generating the user-configurable trait table includes receiving the context-specific static features from a user.

The details of one or more implementations of the disclosure are set forth in the accompanying drawings and the description below. Other aspects, features, and advantages will be apparent from the description and drawings, and from the claims.

### DESCRIPTION OF DRAWINGS

FIG. 1 is a schematic view of an example system for transforming Fast Healthcare Interoperability Resources (FHIR) data.

FIG. 2A is a schematic view of an events table and a traits table.

FIG. 2B is a schematic view of an exemplary events table.

FIG. 2C is a schematic view of an exemplary traits table.

FIG. 3 is a schematic view of a model trainer.

FIG. 4 is a schematic view of components of an exemplary model.

FIG. 5 a flowchart of an example arrangement of operations for a method of transforming FHIR data.

FIG. 6 is a schematic view of an example computing device that may be used to implement the systems and methods described herein.

Like reference symbols in the various drawings indicate like elements.

### DETAILED DESCRIPTION

Metrics for healthcare patients over time (e.g., regular readings of blood pressure, heart rate, sodium/glucose levels, etc.) are routinely used by clinicians to identify at-risk persons. As sensors get more numerous and more data is shared across institutions, clinicians have to sift through increasing amounts of data to understand the trends and identify the probability of “individualized” patient outcomes. Additionally or alternatively, hospital administrators are tracking operational and quality of care metrics such as length of stays, supply of equipment, staffing levels, etc. The end goal is to calculate the probability of a future positive or negative outcome such that timely interventions can be implemented.

Implementations herein include a data transformer to mitigate the time-consuming burden of organizing data by providing a platform to, for example, predict the probability of an outcome (e.g., a health outcome) of a user (e.g., a patient) based on longitudinal patient records (LPR) associated with the user or patient. Clinicians and administrators may use the data transformer as a tool to help prioritize attention with less time devoted to data analysis. The data transformer provides a solution for training machine learning (ML) models using data from an institution's patient population or hospital metrics. The data transformer may enable a prediction endpoint that can be easily integrated into upstream applications.

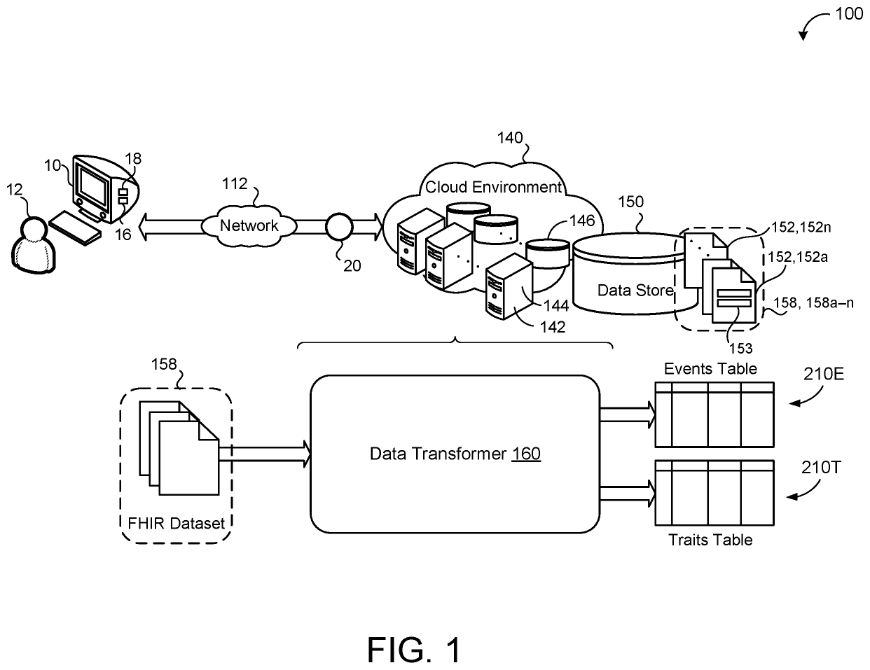

Referring to FIG. 1, in some implementations, an example data transformation system 100 includes a remote system 140 in communication with one or more user devices 10 via a network 112. The remote system 140 may be a single computer, multiple computers, or a distributed system (e.g., a cloud environment) having scalable/elastic resources 142 including computing resources 144 (e.g., data processing hardware) and/or storage resources 146 (e.g., memory hardware). A data store 150 (i.e., a remote storage device) may be overlain on the storage resources 146 to allow scalable use of the storage resources 146 by one or more of the clients (e.g., the user device 10) or the computing resources 144. The data store 150 is configured to store one or more records 152, 152a—n within one or more datasets 158, 158a—n. For example, the records 152 include health data records (i.e., health data) in a Fast Healthcare Interoperability Resources (FHIR) standard format. The records 152 may be grouped together into any number of datasets 158. The data store 150 may be a FHIR data store. In some examples, the records 152 are in a comma-separated value (CSV) format, however the records 152 may be stored in any suitable format.

The remote system 140 may be configured to receive a data transformation query 20 from a user device 10 associated with a respective user 12 via, for example, the network 112. The user device 10 may correspond to any computing device, such as a desktop workstation, a laptop workstation, or a mobile device (i.e., a smart phone). The user device 10 includes computing resources 18 (e.g., data processing hardware) and/or storage resources 16 (e.g., memory hardware). The user 12 may construct the query 20 using a Structured Query Language (SQL) interface. The query 20 may request that the remote system 140 process some or all of the datasets 158 in order to, for example, train one or machine learning models using data from the datasets 158. The trained machine learning models may be used to make predictions based on the training data (e.g., to predict a health outcome for a patient).

The remote system 140 executes a data transformer 160. The data transformer 160 obtains a dataset 158 that includes, for example, health data in the FHIR standard. In other examples, the dataset 158 includes different electronic health data (EHR). In some examples, the remote system 140 retrieves the dataset 158 from the data store 150 or receives the dataset 158 from the user device 10. The query 20 may include a data source of the dataset 158 (e.g., the data store 150). The data transformer 160, in response to determining the data source from the query 20, retrieves the dataset 158 from the data source. The dataset 158 includes a number of healthcare events 153 for one or more patients. For example, the healthcare events 153 may include doctor visits or other appointments, admission details, procedures, tests, measurements (e.g., vital signs), diagnoses, medications and prescriptions, etc. Each event 153 includes data describing or otherwise quantifying the event (e.g., date and times, description and values of vitals, medications, test results, etc.). The healthcare events 153 may include tabular coded numeric and text data (e.g., EHR data), imaging data (e.g., coded images), genomics (e.g., coded sequences and positional data), social data, and/or wearables data (e.g., high frequency waveforms, tabular coded numeric data, etc.).

The FHIR health data of the dataset 158, in some implementations, includes nested data. Health data stored using the FHIR standard is typically in a highly nested format that allows repeated entries at different levels. Because many models (e.g., machine learning model) typically require “flat” (i.e., data that is not nested) data as input, machine learning models generally cannot properly learn from standard FHIR data. To be useful, the data must first be “flattened.” However, machine learning practitioners often struggle with flattening this data efficiently and in a standard manner that is reusable across multiple use cases. Other types of data, such as EHR data, are also generally not “ML ready.” For example, EHR data is often sparse, heterogeneous, and imbalanced.

The data transformer 160, using the FHIR dataset 158, generates an events table 210E that includes each of the healthcare events 153 of the dataset 158. The events table 210E is indexed, in some implementations, by time (i.e., the point in time that the event occurred) and/or a unique identifier (ID) per patient encounter. The events table 210E may include columns that include a time an event 153 occurred, a code for the event 153, one or more values associated with the event, units of the values, etc. The data transformer 160, using the FHIR dataset 158, also generates a traits table 210T. The traits table 210T, like the events table 210E, may be indexed by the unique ID per patient encounter. The traits table columns associated with an ID of a patient, an encounter ID, a gender of the patient, a birth date of the patient, an admission code of the patient, or other columns that describe or define traits of the patient associated with the patient ID. As discussed in more detail below, the remote system 140 may use the events table 210E and the traits table 210T to assist a number of downstream applications. For example, the remote system 140 may use the “flattened” data of the events table 210E and the traits table 210T to train one or more machine learning models. The trained machine models may be used for making predictions, such as for predicting a health outcome for a patient. The events table 210E and the traits table 210T preserves the dataset 158 in a manner that is reusable across many different use cases by persisting the dataset 158 as sequential data (e.g., sequence of labs, vitals, procedures, medications, etc.) into a structured time-series.

In some implementations, the data transformer 160 generates a user-configurable trait table that includes context-specific static features indexed by the unique ID per patient encounter. The data transformer 160 may receive, via the user device 10, the context-specific static features from the user 12. The user-configurable trait table allows user 12 to inject their own context-specific static features that are keyed using the same patient encounters as the events table 210E and the traits table 210T.

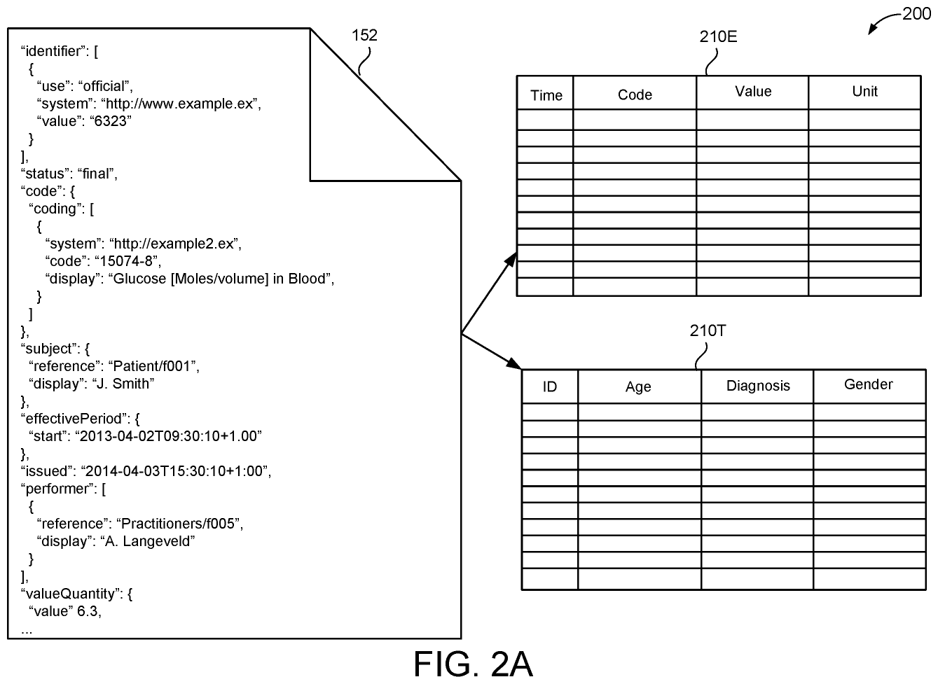

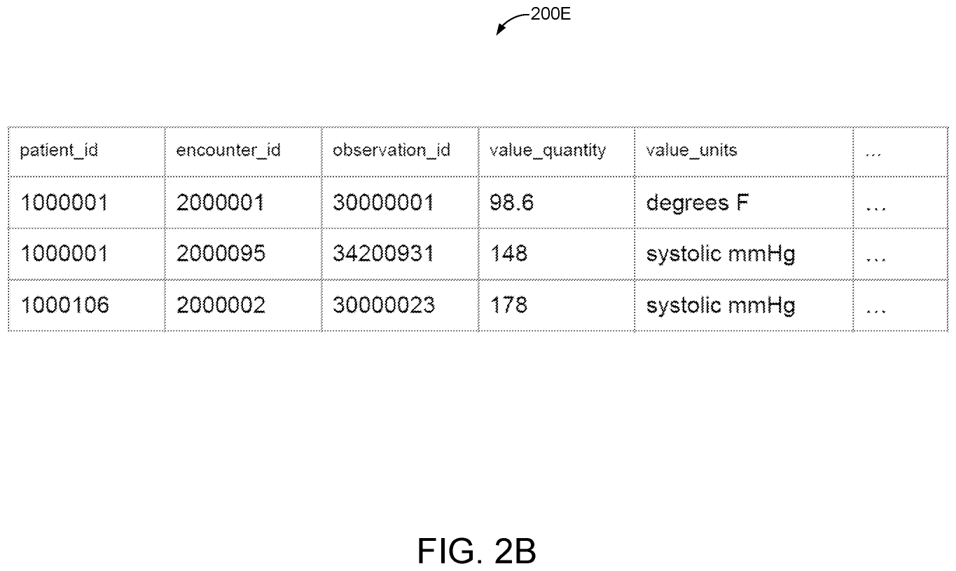

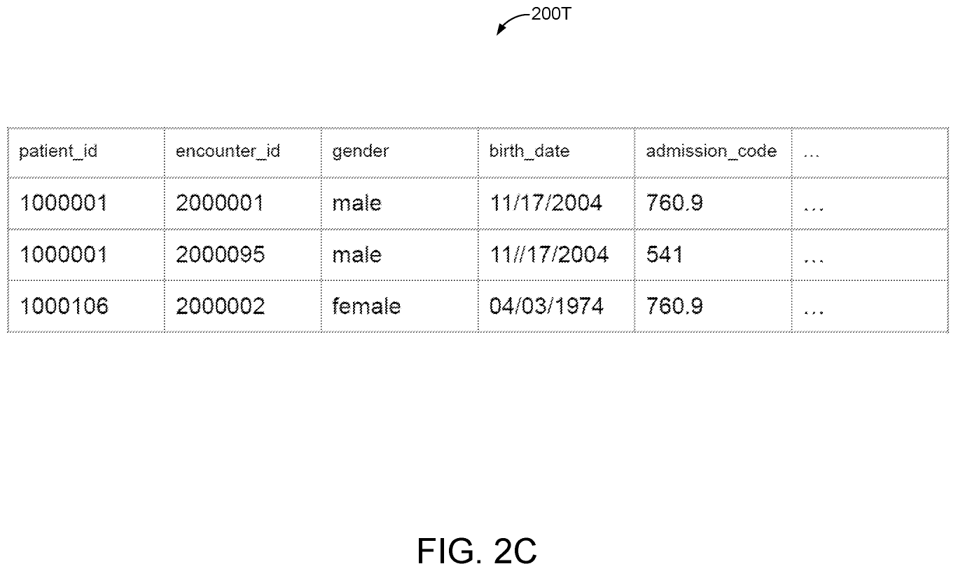

Referring now to FIG. 2A, a schematic view 200 includes an exemplary health record 152 in the FHIR standard. The data of the record 152 is nested and generally unusable for most models in such a nested format, as the models require the data to be flat. The data transformer 160 transforms each record 152 of the dataset 158 into the events table 210E and the traits table 210T. Here the events table 210E as multiple columns that include a “time” column (i.e., a time when the event 153 occurred or was recorded), a “code” column defining or identifying the event (e.g., an encounter ID identifying a temperature reading of a patient), a “value” column (e.g., 98.6 for the temperature reading), and a “unit” column (e.g., degrees Fahrenheit for the temperature reading). These columns are merely exemplary and the events table 210E may include different and/or additional columns depending on the dataset 158 and the desired use cases of the data. For example, the events table 210E may include a patient ID column identifying the patient. The events table 210E represents the dataset 158 as a structured time-series. FIG. 2B includes an exemplary events table 210E with columns associated with a patient ID, an encounter ID, an observation ID, a value, and a value unit. In this example, there are two rows with the same patient ID, as the same patient is associated with two different encounter IDs (e.g., two different visits or tests).

The traits table 210T also includes a number of columns. The traits table 210T includes generally static data (or at least data that is less dynamic that the data of the events table 210E) such as patient demographics (e.g., age, gender, height, weight, etc.). Here, the traits table 210T includes an ID column. The ID column may correspond to the code column of the events table 210E. The traits table 210T also includes an age column, a diagnosis column, and a gender column, however these columns are merely exemplary and the traits table 210T may include any appropriate columns. For example, the traits table 210T includes a patient ID column, an admission code, etc. FIG. 2C includes an exemplary traits table 210T with columns for patient ID, encounter ID, gender, birth data, and admission code. Here, there are two rows with the same patient ID, but each row has a different encounter ID, thus signifying the same patient had two different encounters.

In some implementations, the data transformer, when generating the events table 210E and/or the traits table 210T, normalizes one or more codes, units, numerical data, or any other aspect of the dataset 158 into machine learning-friendly formats. For example, the code “US” may be normalized to “ultrasound” or a pounds unit (i.e., lbs) may be normalized to kilograms.

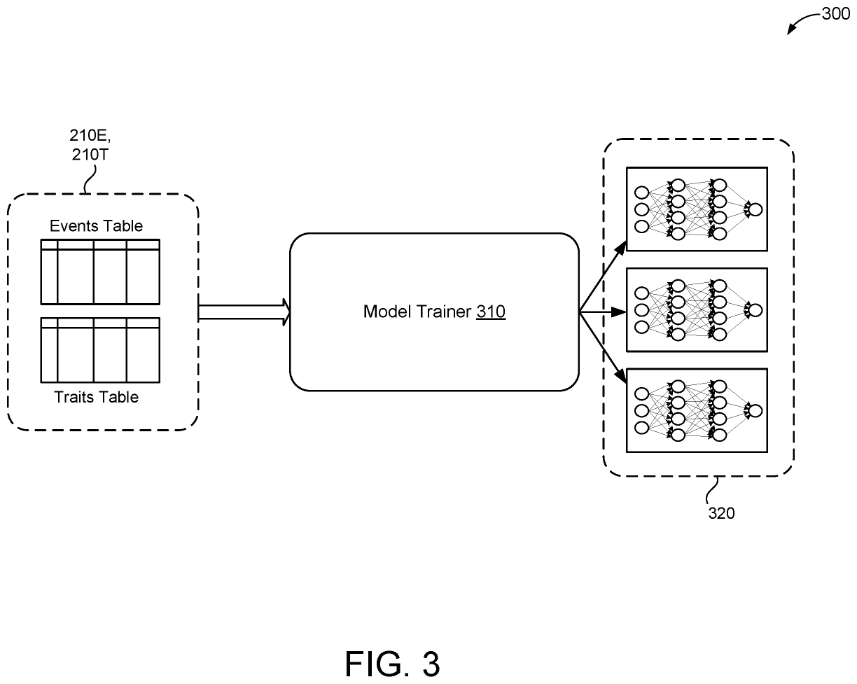

Referring now to FIG. 3, in some implementations, the events table 210E and the traits table 210T are used (e.g., by the remote system 140) to train one or more machine learning models 320. Here, a schematic view 300 includes a model trainer 310 that receives the events table 210E and the traits table 210T and trains multiple machine learning models 320. Each model 320 may be trained to make different predictions based on the training. For example, one model 320 predicts a prognosis for a patient given the event history of the patient.

In some examples, the model 320 is a multi-task model that is trained, using the events table 210E and the traits table 210T, to simultaneously predict outcomes and forecast observation values. That is, because such health records often suffer from severe label imbalance (i.e., the distribution of labels in the training data is skewed) and because labels may be rare, delayed, and/or hard to define, a multi-task model is advantageous. For example, the multi-task model provides a signal boost from high-data nearby problems, is semi-supervised, naturally fits outcomes from time series, and provides additional model evaluation information.

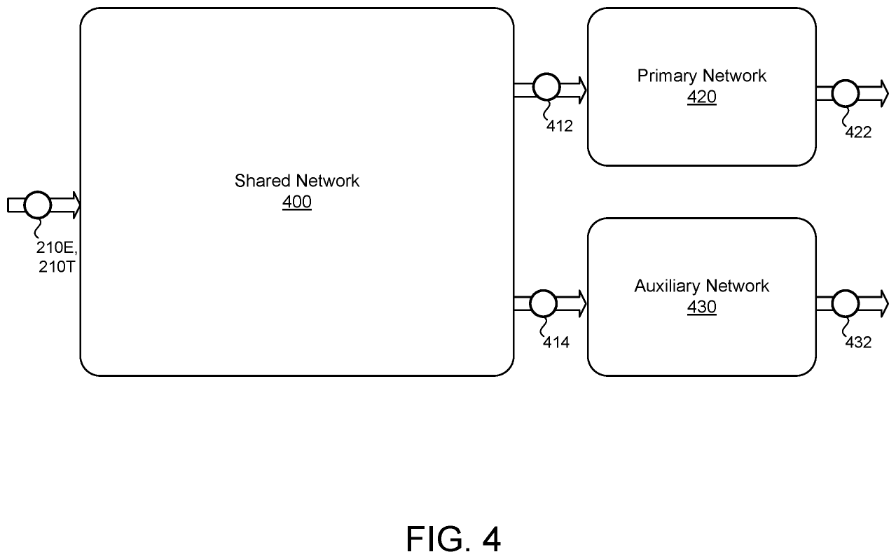

Referring now to FIG. 4, in some implementations, the model 320 includes a shared network 400, a primary network 420, and an auxiliary network 430. The shared network 400 receives the event table 210E and the traits table 210T. The shared network 400, in some examples, includes an encoder (such as a long short-term memory (LSTM) encoder). The encoder distills the data from the tables 210E, 210T into a lower-dimensional representation. The shared network 400 generates a first output 412 for the primary network 420 and a second output 414 for the auxiliary network 430. The primary network 420, using the first output 412 from the shared network 400, predicts an outcome 422 (e.g., a health outcome for a patient). In some examples, the primary network 420 includes a classifier (e.g., a dense layer on top of an encoder output) to predict the outcome 422. The auxiliary network 430, using the second output 414 from the shared network 400, predicts or forecasts a time-series 432 for observation values. In some implementations, the time-series is an LSTM rollout of the structured time-series with masked loss. The auxiliary network 430 may include a decoder (e.g., an autoregressive LSTM model) that produces fixed-interval predictions for the multivariate times-series event data. The networks 400, 420, 430 may be co-trained (e.g., via the model trainer 310) with a weighted sum loss.

After the model 320 is trained, a user 12 may request a prediction via a prediction request that includes events and traits for a particular patient similar to the data the model 320 was trained on. The user may provide the data in, for example, the FHIR format and the system 100 may automatically flatten the data into the events table 210E and the traits table 210T for processing by the model 320. In other examples, the prediction request includes the data pre-processed in a format suitable for the model 320. Using the provided data, the model 320 predicts a health outcome 422. Optionally the model 320 additionally forecasts one or more observation values via a time-series 432.

In some implementations, the model trainer 310 trains the model 320 in response to a request. For example, the request 20 may include a request to train a model 320 to predict one or more specific health outcomes 422. In response to the request, the system 100 generates the events table 210E and the traits table 210T from the data specified by, for example, the request (e.g., FHIR data or any other repository). The system 100 may select a cohort from the data to train the model 320. The system may select the cohort based on the request 20 (i.e., based on the health outcomes 422 desired for prediction). For example, a user may request a model 320 to predict a likelihood of a health outcome 422 (e.g., death, illness, discharge, etc.) within three days of admission to a hospital. In this example, the system 100 may ensure that the cohort to train the model 320 only includes patient records where the discharge date is more than two days after admission. The user 12 and/or the system 100 may generate or tailor the cohort used to train the model 320 based on the health outcome 422 to be predicted. For example, the user 12 may submit a query or request to the system 100 that includes a number of parameters defining the health outcome 422. Accordingly, the user 12 (i.e., via the user device 10) and/or the system 100 may query or filter the data records 152 to obtain the data records 152 relevant for the desired health outcome 422.

In some implementations, the model 320 may be trained to predict multiple different health outcomes simultaneously. For example, the model 320 includes two or more different output layers that each provides a respective classification result for a respective health outcome 422.

Thus, implementations herein include a data transformation system 100 that persists sequential data (e.g., sequence of labs, vital measurements, procedures, medications, etc.) into a structured time-series via intermediate event tables 210E and traits tables 210T. The events table 210E may capture events and is indexed by time and a unique ID for a patient encounter. The traits table 210T may capture relatively static data such as patient demographics. The system 100 may normalize the data (e.g., codes, units, etc.) into formats compatible for machine learning. The system 100 provides a tabular schema that users can, in addition to training a machine learning model, use to aggregate and slice segments of data for insights, anomaly detection, etc. The system allows for the injection of external data (e.g., data representing context-specific static features keyed by a particular patient encounter). Models trained on the event table 210E and traits table 210T may predict the probability of an outcome based on longitudinal patient records. These predictions allow clinicians and administrators to prioritize without having to spend significant amounts of time on data analysis.

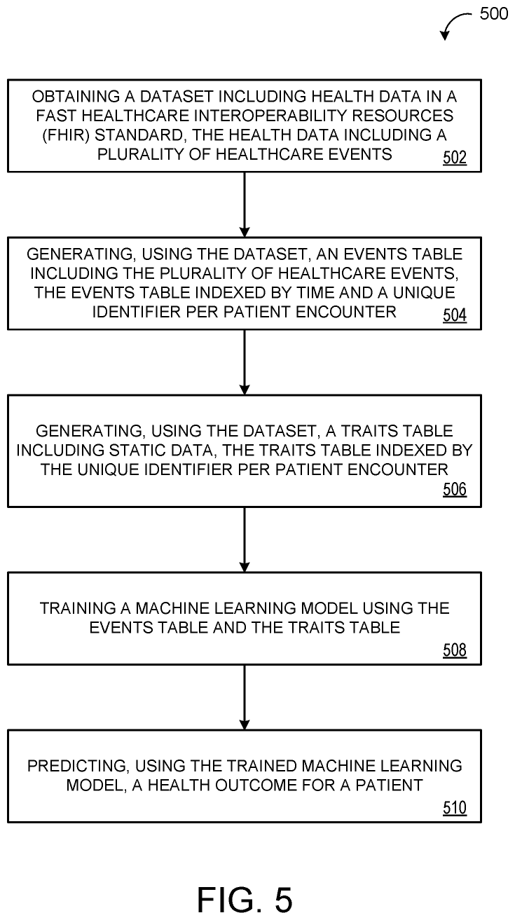

FIG. 5 is a flowchart of an exemplary arrangement of operations for a method 500 of transforming data. The computer-implemented method 500, when executed by data processing hardware 144 causes the data processing hardware 144 to perform operations. The method 500, at operation 502, includes obtaining a dataset 158 that includes health data records 152 in a Fast Healthcare Interoperability Resources (FHIR) standard. The health data includes a plurality of healthcare events. At operation 504, the method 500 includes generating, using the dataset 158, an events table 210E that includes the plurality of healthcare events. The events table 210E is indexed by time and a unique identifier per patient encounter. At operation 506, the method 500 includes generating, using the dataset 158, a traits table 210T that includes static data. The traits table 210T is indexed by the unique identifier per patient encounter. The method 500, at operation 508, includes training a machine learning model 320 using the events table 210E and the traits table 210T. At operation 510, the method 500 includes predicting, using the trained machine learning model 320 and one or more additional healthcare events associated with a patient, a health outcome 422 for the patient.

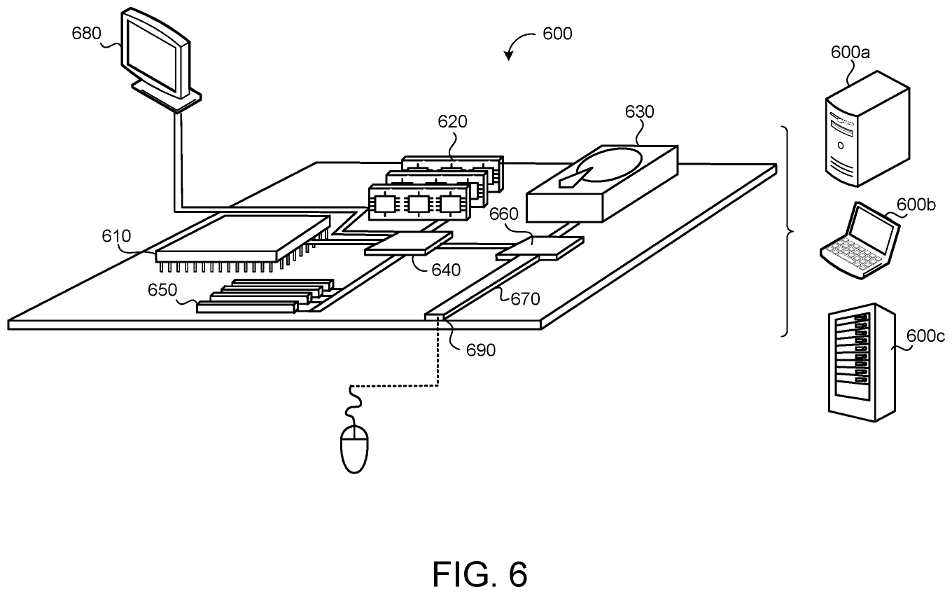

FIG. 6 is a schematic view of an example computing device 600 that may be used to implement the systems and methods described in this document. The computing device 600 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations of the inventions described and/or claimed in this document.

The computing device 600 includes a processor 610, memory 620, a storage device 630, a high-speed interface/controller 640 connecting to the memory 620 and high-speed expansion ports 650, and a low speed interface/controller 660 connecting to a low speed bus 670 and a storage device 630. Each of the components 610, 620, 630, 640, 650, and 660, are interconnected using various busses, and may be mounted on a common motherboard or in other manners as appropriate. The processor 610 can process instructions for execution within the computing device 600, including instructions stored in the memory 620 or on the storage device 630 to display graphical information for a graphical user interface (GUI) on an external input/output device, such as display 680 coupled to high speed interface 640. In other implementations, multiple processors and/or multiple buses may be used, as appropriate, along with multiple memories and types of memory. Also, multiple computing devices 600 may be connected, with each device providing portions of the necessary operations (e.g., as a server bank, a group of blade servers, or a multi-processor system).

The memory 620 stores information non-transitorily within the computing device 600. The memory 620 may be a computer-readable medium, a volatile memory unit(s), or non-volatile memory unit(s). The non-transitory memory 620 may be physical devices used to store programs (e.g., sequences of instructions) or data (e.g., program state information) on a temporary or permanent basis for use by the computing device 600. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

The storage device 630 is capable of providing mass storage for the computing device 600. In some implementations, the storage device 630 is a computer-readable medium. In various different implementations, the storage device 630 may be a floppy disk device, a hard disk device, an optical disk device, or a tape device, a flash memory or other similar solid state memory device, or an array of devices, including devices in a storage area network or other configurations. In additional implementations, a computer program product is tangibly embodied in an information carrier. The computer program product contains instructions that, when executed, perform one or more methods, such as those described above. The information carrier is a computer- or machine-readable medium, such as the memory 620, the storage device 630, or memory on processor 610.

The high speed controller 640 manages bandwidth-intensive operations for the computing device 600, while the low speed controller 660 manages lower bandwidth-intensive operations. Such allocation of duties is exemplary only. In some implementations, the high-speed controller 640 is coupled to the memory 620, the display 680 (e.g., through a graphics processor or accelerator), and to the high-speed expansion ports 650, which may accept various expansion cards (not shown). In some implementations, the low-speed controller 660 is coupled to the storage device 630 and a low-speed expansion port 690. The low-speed expansion port 690, which may include various communication ports (e.g., USB, Bluetooth, Ethernet, wireless Ethernet), may be coupled to one or more input/output devices, such as a keyboard, a pointing device, a scanner, or a networking device such as a switch or router, e.g., through a network adapter.

The computing device 600 may be implemented in a number of different forms, as shown in the figure. For example, it may be implemented as a standard server 600a or multiple times in a group of such servers 600a, as a laptop computer 600b, or as part of a rack server system 600c.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

A software application (i.e., a software resource) may refer to computer software that causes a computing device to perform a task. In some examples, a software application may be referred to as an “application,” an “app,” or a “program.” Example applications include, but are not limited to, system diagnostic applications, system management applications, system maintenance applications, word processing applications, spreadsheet applications, messaging applications, media streaming applications, social networking applications, and gaming applications.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A number of implementations have been described. Nevertheless, it will be understood that various modifications may be made without departing from the spirit and scope of the disclosure. Accordingly, other implementations are within the scope of the following claims.

What is claimed is:

1. A computer-implemented method executed by data processing hardware that causes the data processing hardware to perform operations comprising:
obtaining a dataset comprising health data in a Fast Healthcare Interoperability Resources (FHIR) standard, the health data comprising a plurality of healthcare events;
generating, using the dataset, an events table comprising the plurality of healthcare events, the events table indexed by time and a unique identifier per patient encounter;
generating, using the dataset, a traits table comprising static data, the traits table indexed by the unique identifier per patient encounter;
training a machine learning model using the events table and the traits table; and
predicting, using the trained machine learning model and one or more additional healthcare events associated with a patient, a health outcome for the patient.

2. The method of claim 1, wherein obtaining the dataset comprises:
receiving a training request defining a data source of the dataset; and
retrieving the dataset from the data source.

3. The method of claim 1, wherein the operations further comprise normalizing one or more codes of the health data.

4. The method of claim 1, wherein the operations further comprise normalizing one or more units of the health data.

5. The method of claim 1, wherein the dataset comprises a comma-separated values file.

6. The method of claim 1, wherein the traits table comprises patient demographics.

7. The method of claim 1, wherein the events table represents the dataset as a structured time-series.

8. The method of claim 1, wherein the dataset comprises nested data.

9. The method of claim 1, wherein the operations further comprise generating a user-configurable trait table comprising context-specific static features indexed by the unique identifier per patient encounter.

10. The method of claim 9, wherein generating the user-configurable trait table comprises receiving the context-specific static features from a user.

11. A system comprising:
data processing hardware; and
memory hardware in communication with the data processing hardware, the memory hardware storing instructions that when executed on the data processing hardware cause the data processing hardware to perform operations, the operations comprising:
obtaining a dataset comprising health data in a Fast Healthcare Interoperability Resources (FHIR) standard, the health data comprising a plurality of healthcare events;
generating, using the dataset, an events table comprising the plurality of healthcare events, the events table indexed by time and a unique identifier per patient encounter;
generating, using the dataset, a traits table comprising static data, the traits table indexed by the unique identifier per patient encounter;
training a machine learning model using the events table and the traits table; and
predicting, using the trained machine learning model and one or more additional healthcare events associated with a patient, a health outcome for the patient.

12. The system of claim 11, wherein obtaining the dataset comprises:
receiving a training request defining a data source of the dataset; and
retrieving the dataset from the data source.

13. The system of claim 11, wherein the operations further comprise normalizing one or more codes of the health data.

14. The system of claim 11, wherein the operations further comprise normalizing one or more units of the health data.

15. The system of claim 11, wherein the dataset comprises a comma-separated values file.

16. The system of claim 11, wherein the traits table comprises patient demographics.

17. The system of claim 11, wherein the events table represents the dataset as a structured time-series.

18. The system of claim 11, wherein the dataset comprises nested data.

19. The system of claim 11, wherein the operations further comprise generating a user-configurable trait table comprising context-specific static features indexed by the unique identifier per patient encounter.

20. The system of claim 19, wherein generating the user-configurable trait table comprises receiving the context-specific static features from a user.
